(19) United States
(12) Patent Application Publication
(10) Pub. No.: US 2025/0231565 A1
(43) Pub. Date: Jul. 17, 2025

(54) AUTONOMOUS HEALTHCARE ROBOT FOR SECURE CARGO TRANSPORT, TELEVISITS, AND IMAGE CLASSIFICATION

(71) Applicant: Che Martin, New York, NY (US)

(72) Inventor: Che Martin, New York, NY (US)

(21) Appl. No.: 18/949,898

(22) Filed: Nov. 15, 2024

Related U.S. Application Data
(60) Provisional application No. 63/599,074, filed on Nov. 15, 2023.

(51) Int. Cl. G05D 1/43; G05D 1/622; G06F 3/041; G06V 10/764; G07C 9/00; G16H 40/20; G16H 40/40; G16H 80/00; H04N 7/14; H04N 23/69

(52) U.S. Cl. CPC G05D 1/43; G05D 1/622; G06F 3/0412; G06V 10/764; G07C 9/00182; G16H 40/20; G16H 40/40; G16H 80/00; H04N 7/141; H04N 23/69

(57) ABSTRACT

An autonomous healthcare robot system provides secure cargo transport, televisit capabilities, and AI-based image classification in space-restricted environments. The robot comprises a mobile base with omnidirectional drive, a cylindrical body housing a secure cargo compartment, multiple sensors for navigation, a touchscreen interface, and cameras for televisits and image capture. The compact design allows operation in areas as narrow as 33 inches. An onboard computer controls autonomous navigation, multi-factor authenticated cargo access, televisits, and image classification tasks. The modular, multi-purpose design enables efficient use of healthcare staff and minimizes unnecessary person-to-person contact.

### CROSS-REFERENCE TO RELATED APPLICATIONS

This application claims the benefit of U.S. Provisional Patent Application No. 63/599,074, filed on Nov. 15, 2023, entitled “A NOVEL SIZE-APPROPRIATE AUTONOMOUS ROBOT TO FACILITATE CARE TELEVISITS, CLASSIFICATION TASKS AND SECURE SAMPLE TRANSPORT,” the entire contents of which are hereby incorporated by reference.

### STATEMENT REGARDING FEDERALLY SPONSORED RESEARCH OR DEVELOPMENT

Not Applicable

### FIELD OF THE INVENTION

The present invention relates generally to mobile robots, and more specifically to autonomous navigation robots used in healthcare environments with secure cargo compartments for transporting objects between locations.

### BACKGROUND

Healthcare facilities face several key challenges today: (1) inefficient use of staff for mundane transport tasks, (2) the need to limit person-to-person contact to mitigate pathogen spread, and (3) difficulty implementing AI-driven classification tasks like PPE monitoring. Existing autonomous robot solutions address some of these issues, but have limitations. Many are too large to operate in restricted spaces like clinical labs. Others lack the versatility to perform multiple functions like secure transport, televisits, and image classification within a single device. There is a need for a compact, multi-purpose autonomous robot that can navigate tight spaces while providing secure cargo transport, televisit capabilities, and AI-based image classification in healthcare settings.

### SUMMARY

The present invention provides a multipurpose autonomous robot system for use in healthcare facilities or other applicable industries (e.g. Laboratories). In some embodiments, the robot system comprises:
a mobile base with an omnidirectional drive system;
a generally cylindrical body mounted on the mobile base;
a secure cargo compartment integrated into the cylindrical body;
a plurality of sensors for autonomous navigation and obstacle avoidance;
a touchscreen user interface mounted on an upper portion of the cylindrical body;
a camera system for facilitating televisits and image capture;
an onboard computer system for controlling robot functions; and
a wireless communication interface.

The robot system is configured to autonomously navigate in space-restricted environments to securely transport cargo between locations, facilitate televisits, and perform AI-based image classification tasks. The compact design allows operation in areas as narrow as 33 inches wide.

In some embodiments, the omnidirectional drive system comprises three omni-wheels positioned 120° apart. In other embodiments, four mecanum wheels may be used. The plurality of sensors may include a 360-degree LIDAR sensor, a depth camera, inertial measurement units (IMUs sensors), and ultrasonic or infrared proximity sensors. The sensors enable simultaneous localization and mapping (SLAM) for autonomous navigation. Access to the secure cargo compartment may require multi-factor authentication, such as RFID card plus PIN code entry on the touchscreen. The cargo compartment may be modular and swappable to accommodate different cargo configurations. The camera system may include a web camera for televisits and a depth camera for navigation and image classification tasks. The onboard computer can run AI models for tasks like PPE compliance monitoring or inventory checking.

These and other aspects of the invention will become apparent from the following detailed description, which is meant to illustrate, but not limit, the invention.

### BRIEF DESCRIPTION OF THE DRAWINGS

The features and advantages of the present disclosure will become more apparent from the detailed description set forth below when taken in conjunction with the drawings. The accompanying drawings, which are incorporated in and constitute a part of this specification, illustrate several aspects of the invention and together with the description, serve to explain the principles of the invention.

FIG. 1 shows an isometric front view of an exemplary embodiment of the robot.

FIG. 2 illustrates a front view of an exemplary embodiment depicting multiple sensors used for navigation and obstacle avoidance.

FIG. 3 shows a left side view of an exemplary embodiment depicting key components.

FIG. 4 illustrates a right side view of an exemplary embodiment showing power and charging components.

FIG. 5 presents a top view of an exemplary embodiment showing a top-loading cargo compartment.

FIG. 6 shows a rear view of an embodiment with a rear-loading cargo compartment.

FIG. 7 illustrates the sensor fields of view for an exemplary embodiment.

FIGS. 8A and 8B depict various maneuvering operations of an exemplary embodiment.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others.

Further, it is to be understood that other embodiments may be utilized. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents.

### DETAILED DESCRIPTION

The present invention can be understood more readily by reference to the following detailed description of the invention and the examples included therein. Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this present disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings. Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the present disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the present disclosure. Thus, if there are a variety of additional steps that can be performed it is understood that each of these additional steps can be performed with any specific aspect or combination of aspects of the methods of the present disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

All publications mentioned herein are incorporated herein by reference to disclose and describe the methods and/or materials in connection with which the publications are cited.

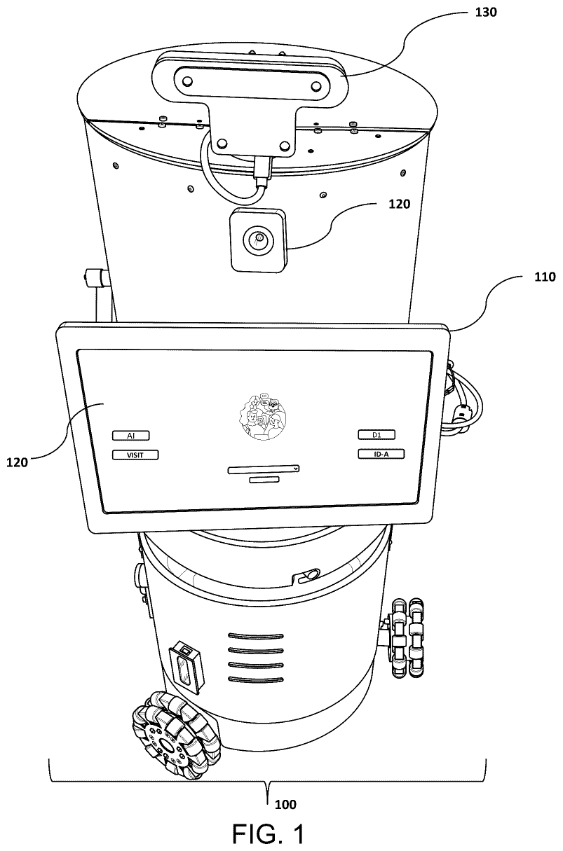

Referring to FIG. 1, an autonomous healthcare robot 100 is shown according to an exemplary embodiment of the present invention. The robot 100 comprises a generally cylindrical body mounted on a mobile base. A touchscreen user interface 110 is mounted on an upper front portion of the cylindrical body. The touchscreen 110 displays a customized interactive user interface menu 120 for controlling robot functions. A depth camera 130 is also mounted on the upper front portion for navigation and image capture.

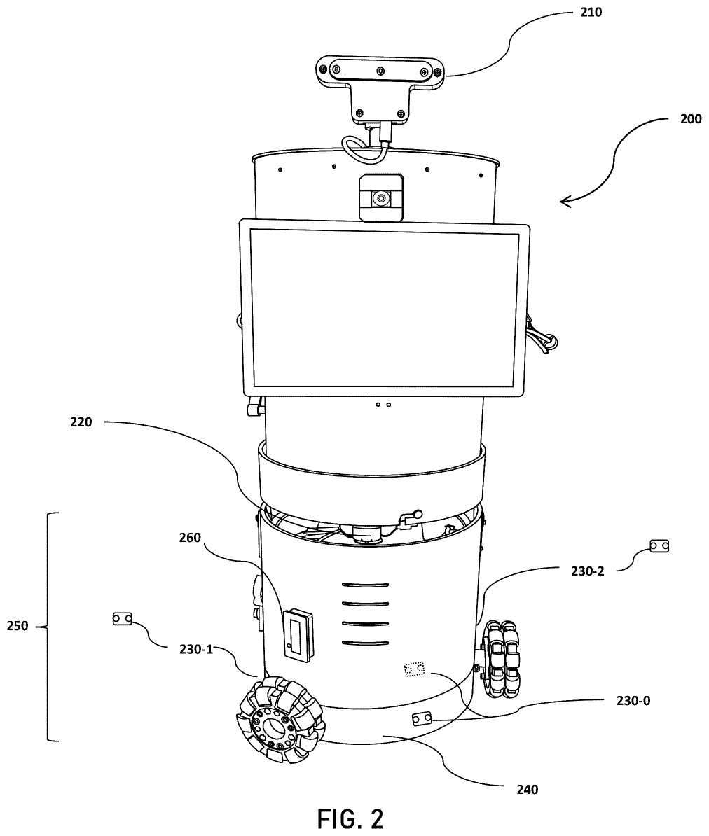

FIG. 2 illustrates the multiple sensors used for autonomous navigation and obstacle avoidance in an exemplary embodiment 200. A depth camera 210 provides obstacle detection within an approximately 72 degree field of view from the front of the robot. A 360-degree LIDAR sensor 220 detects obstacles up to 6 meters away with a 360° field of view around its mount point on the robot base 250. Additional proximity sensors 230-0, 230-1, 230-2 such as infrared or ultrasonic sensors may be included in the robot base cap 240 or robot base 250 to further enhance obstacle detection capabilities.

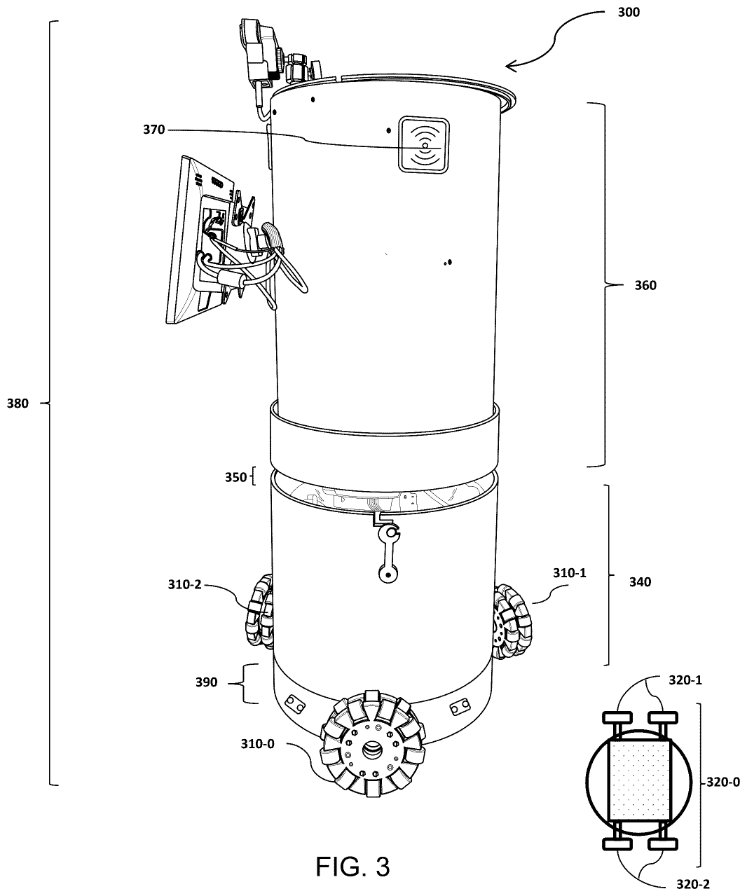

Referring to FIG. 3, an exemplary embodiment 300 achieves omnidirectional movement using three omni-wheels 310-0, 310-1, 310-2 positioned 120° apart. In some embodiments, four mecanum wheels may be used instead, with two left wheels 320-1 and two right wheels 320-2 on opposite sides of the robot body. The robot base 340 houses the drive system, battery, and control electronics. A Mecanum wheel is an omnidirectional wheel design for a land-based vehicle to move in any direction. A transparent sensor window 350 allows operation of the sensors. The cargo-carrying portion 360 is supported above the base. In some embodiments, the robot may have a height of approximately 27.5 inches 380 and a base diameter of approximately 12.5 inches, allowing navigation in restricted areas as narrow as 33 inches.

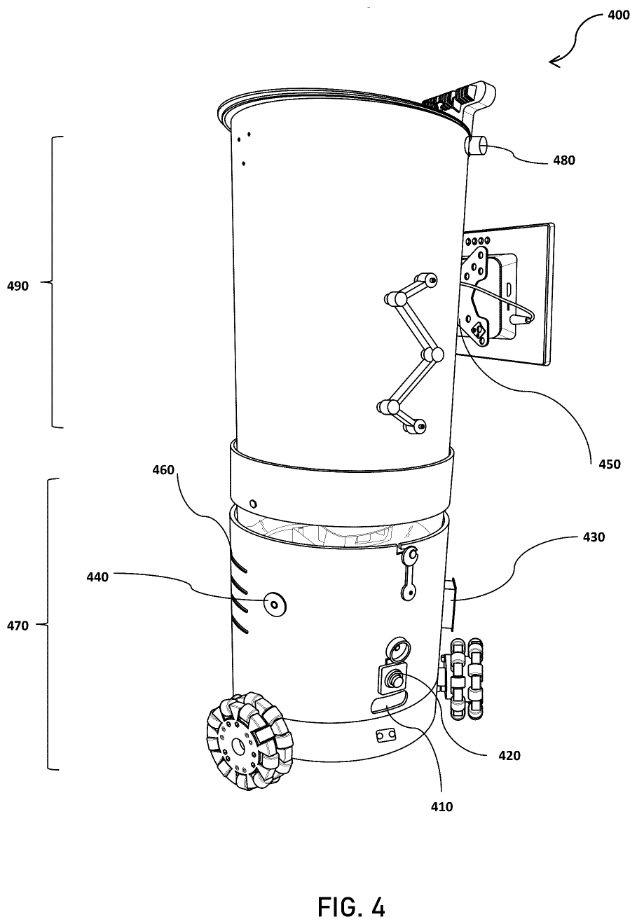

FIG. 4 shows power-related components in an exemplary embodiment 400. A battery meter 430 allows monitoring of battery charge level. A charging port 440 enables battery recharging. The robot base 470 houses the main power switch 420 and system power supply switch 410. Vents 460 facilitate heat dissipation. In some embodiments, the touchscreen may automatically tilt up 10 degrees via a servo-powered hinge 450 in response to hand gestures detected by a motion sensor 480.

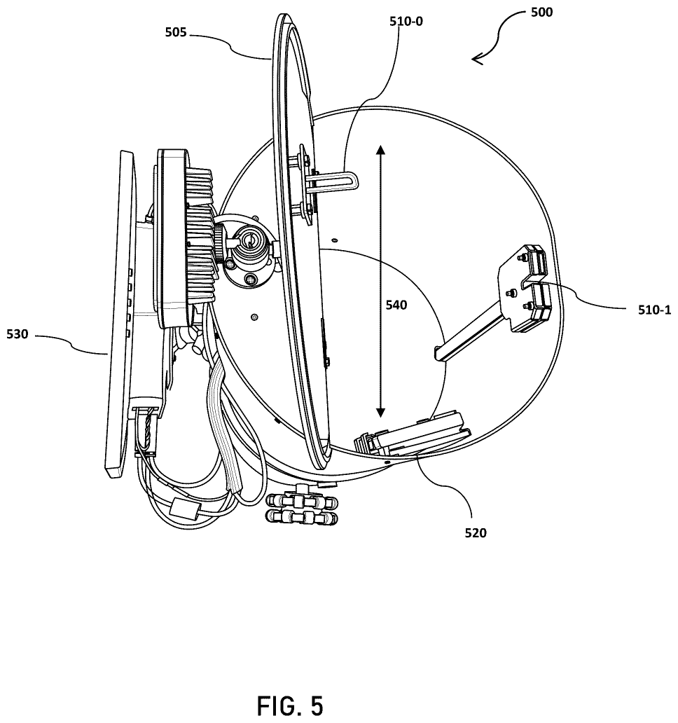

FIG. 5 illustrates a top-loading cargo compartment in an exemplary embodiment 500. The cargo area is secured by a lid 505 and electronic locking system 510-0, 510-1. An RFID reader 520 and touchscreen PIN entry 530 provide multi-factor authentication for cargo access. The cargo bay 540 may be approximately 10 inches wide and able to carry up to 15 pounds.

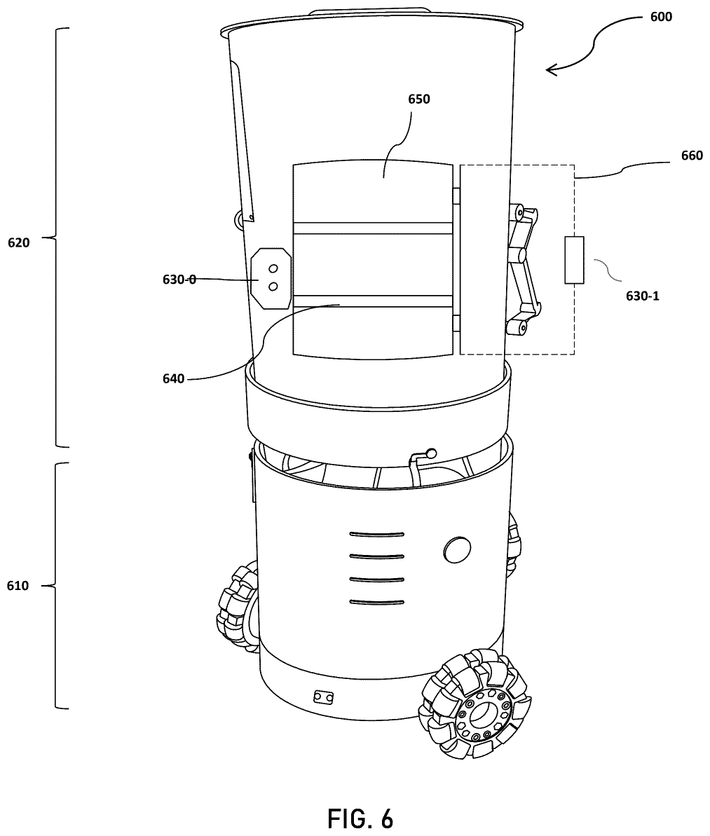

FIG. 6 shows an alternative rear-loading cargo compartment embodiment 600. Access is via a rear door 660 secured by an electronic lock 630-0, 630-1. The cargo area 650 may contain modular shelving to accommodate different cargo configurations.

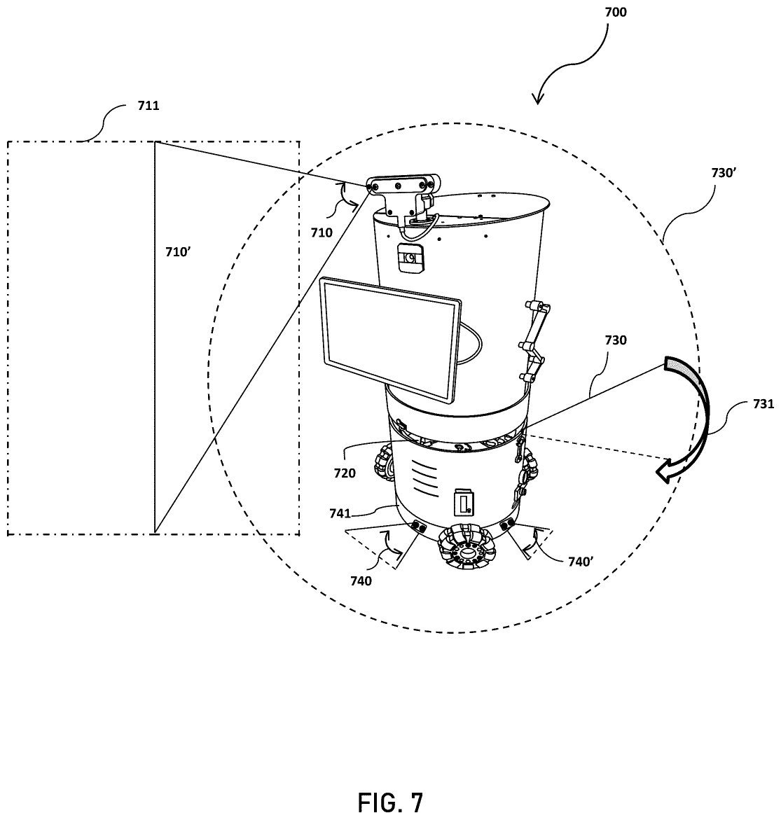

FIG. 7 depicts sensor fields of view in an exemplary embodiment 700. The depth camera has a vertical field of view 710, 710′ and horizontal field of view 711. The 360-degree LIDAR 720 provides a 360° detection field 730, 730′. Additional proximity sensors 740, 740′ in the base cap 741 provide short-range detection.

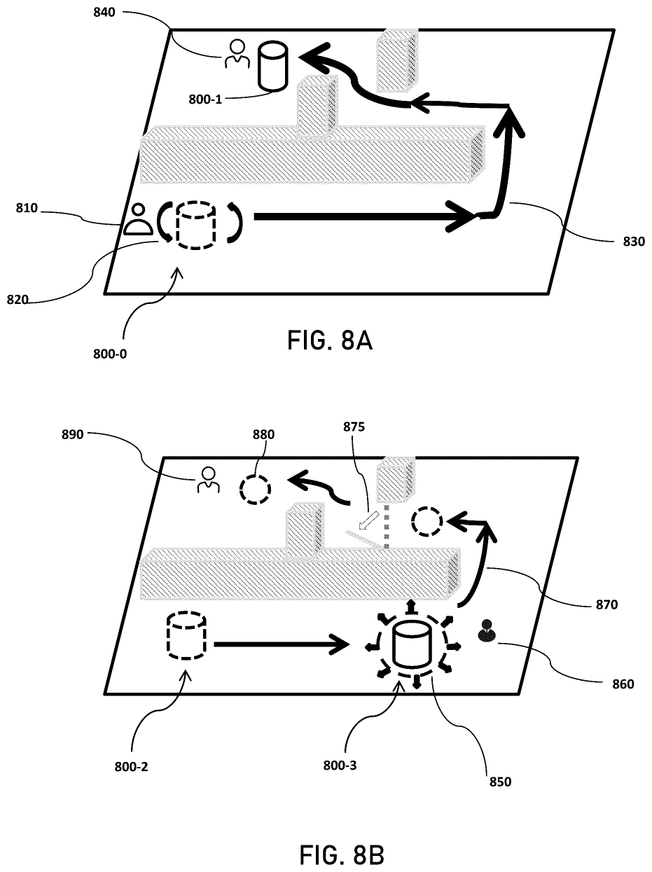

FIGS. 8A and 8B illustrate autonomous navigation capabilities. In FIG. 8A, the robot 800-0 receives a destination input from a sender 810, performs an in-place rotation 820, then navigates 830 to the destination 800-1 to meet a recipient 840. FIG. 8B shows the robot 800-2 detouring 870 around an obstacle 860 while following a delivery route. The robot's omnidirectional capabilities allow flexible maneuvering 850 to avoid obstacles. Additional modules may enable the robot to interact with automatic doors 875 or elevator systems.

In operation, the robot can be controlled locally via the touchscreen interface or remotely through a wireless network connection. Users can instruct the robot to autonomously navigate to predefined destinations, access the secure cargo area, initiate televisits, or switch to image classification mode. Administrative functions like adding destinations, managing authentication methods, and updating system parameters can be performed remotely.

In one or more embodiments, for secure cargo transport, users authenticate using RFID and PIN entry to access the cargo compartment. The robot then autonomously navigates to the specified destination, avoiding obstacles using its sensor array and SLAM algorithms. Gentle acceleration and deceleration help protect sensitive cargo like biological samples. In one or more instances, televisits may be facilitated using the touchscreen, built-in webcam, speakers, and industry-standard conferencing software. The robot can autonomously navigate to a patient's location to enable remote provider-patient interaction. When not navigating, the robot can perform AI-based image classification tasks using its depth camera and onboard computer. Example applications include PPE compliance monitoring, inventory checking, or gathering image data to develop clinical decision support tools.

In one or more embodiments, the modular design allows the cargo compartment to be swapped to accommodate different delivery needs. Additional modules can be added to enhance functionality, such as environmental sensors or alternative power sources. The robot's omnidirectional movement capabilities may allow for flexible maneuvering in tight spaces. As illustrated in FIG. 8A, the robot 800-0 may receive a destination input from a sender 810. The robot can then perform an in-place rotation 820 before navigating 830 to the specified destination 800-1 to meet a recipient 840. This ability to rotate in place and move in any direction enables efficient navigation in space-restricted environments. FIG. 8B further demonstrates the robot's obstacle avoidance capabilities. While following a delivery route, the robot 800-2 may encounter an obstacle 860. In response, the robot can execute a detour 870 around the obstacle, leveraging its omnidirectional capabilities to perform flexible maneuvering 850. This adaptive navigation ensures safe and efficient operation in dynamic healthcare environments.

In some embodiments, the robot may be equipped with additional modules that enable interaction with building systems. For example, the robot may be capable of sending signals to elevator systems and automatic doors 875, allowing them to open upon approach. This functionality facilitates uninterrupted movement along delivery routes, even when traversing multiple floors or passing through secured areas.

In one or more embodiments, the robot's onboard computer system may be configured leverage information from sensors such as IMUs and LIDAR with simultaneous localization and mapping (SLAM) algorithms for autonomous navigation. These algorithms enable the robot to construct and update a map of its environment while simultaneously tracking its position within that map. This capability allows the robot to navigate effectively in both known and unknown environments, adapting to changes in the facility layout over time.

In certain embodiments, the robot may be part of a larger fleet managed by a centralized system. A fleet management system, such as Open-RMF (Open Robotics Middleware Framework), may be employed to coordinate multiple robot units within a healthcare facility. This system can optimize task allocation, manage traffic, and ensure efficient utilization of the robot fleet across various departments and functions.

In one or more embodiments, the robot's AI-based image classification capabilities can be leveraged for various tasks when the robot is not actively navigating. For example, the robot may be used for PPE compliance monitoring, scanning areas to detect whether individuals are wearing appropriate protective equipment such as masks. In inventory management applications, the robot can be deployed to check stock levels in supply rooms, using its cameras and AI models to identify and count items on shelves.

In one or more embodiments, the modular design of the cargo compartment allows for customization to meet specific delivery needs. For instance, in a clinical laboratory setting, the cargo area may be configured with temperature-controlled compartments for transporting sensitive biological samples. In a pharmacy application, the cargo area might include secure, individually lockable sections for delivering medications to different departments.

In one or more embodiments, the robot's televisit capabilities can be particularly valuable in scenarios where direct person-to-person contact should be minimized, such as during infectious disease outbreaks. Healthcare providers can use the robot to conduct remote consultations with patients, leveraging the onboard camera, display, speakers, and microphone. This functionality can help maintain continuity of care while reducing the risk of pathogen transmission.

In some embodiments, the robot may include environmental sensors to monitor conditions such as temperature, humidity, or air quality as it moves through the facility. This data can be used to identify areas that may require attention from facilities management, ensuring optimal conditions for patient care and sensitive equipment. In one or more embodiments, the robot's compact size and maneuverability make it well-suited for operation in space-restricted areas such as clinical labs, sample receiving rooms, and narrow corridors. Its ability to navigate in spaces as narrow as 33 inches wide allows it to access areas that may be challenging for larger autonomous systems or human personnel pushing carts.

In one or more embodiments, the multi-factor authentication system for accessing the cargo compartment provides a high level of security for transported items. In healthcare settings, this is particularly important for ensuring the integrity and privacy of sensitive materials such as biological samples, medications, or confidential documents. The combination of RFID, PIN entry, and potentially biometric or SMS authentication offers robust protection against unauthorized access.

In one or more embodiments, the robot's ability to integrate with building systems such as elevators and automatic doors enhances its autonomy and efficiency. By communicating with these systems, the robot can navigate seamlessly between floors and through secured areas without human intervention, streamlining operations in multi-story healthcare facilities.

Similarly, the robot may be equipped to communicate with automatic door systems. As the robot approaches a closed automatic door along its navigation path, it may send a signal to trigger the door to open. This functionality may enable smooth and uninterrupted movement through doorways without requiring human assistance. In one or more embodiments, the integration with building systems may be facilitated through additional sensors or communication modules housed within the modular cargo-carrying section or elsewhere on the robot body. For example, an IR transmitter may be installed to send elevator call signals, while an RFID tag reader could be used to access secure areas through RFID-enabled doors.

In some embodiments, the robot may leverage its existing wireless communication capabilities, such as Wi-Fi or Bluetooth, to interface with smart building management systems. This may allow the robot to receive real-time updates on elevator status, door lock conditions, or even adjust its route based on building occupancy data. In one or more embodiments, the robot's navigation system may be programmed to account for the time required for elevators to arrive and doors to open. This may enable efficient path planning that minimizes wait times at these transition points. Additionally, the robot may be configured to communicate its intended path to the building management system, allowing for optimized scheduling of elevator usage across multiple robots in a fleet.

In particular embodiments, the robot may be equipped with a camera or other sensor specifically dedicated to reading floor numbers in elevators or room numbers on doors. This may enhance the robot's ability to confirm it has reached the correct floor or destination, adding an additional layer of accuracy to its navigation capabilities. In one or more embodiments, the robot's integration with building systems may also extend to security features. For instance, the robot may be programmed to only access certain floors or areas based on its current task assignment, adhering to the same access control protocols as human staff members. This may be achieved through a combination of the robot's onboard authentication systems and communication with the building's access control database.

In some implementations, the robot may be capable of providing feedback to the building management system. For example, if the robot encounters a malfunctioning automatic door or an out-of-service elevator, it may send an alert to facilities management. This capability may contribute to improved maintenance and quicker resolution of building system issues. The ability to interact with building systems may significantly expand the robot's operational range within a healthcare facility. It may enable the robot to navigate seamlessly between different floors, departments, and secure areas, enhancing its utility for tasks such as specimen transport, medication delivery, or facilitating inter-departmental televisits.

In one or more instances, the robot system may be configured to navigate in spaces as narrow as 33 inches wide. This capability may be enabled by the compact cylindrical body design with a base diameter of approximately 12.5 inches. The omnidirectional drive system, comprising either three omni-wheels positioned 120° apart or four mecanum wheels, may allow for precise maneuvering in tight spaces. In one or more embodiments, the secure cargo compartment may be integrated into the cylindrical body of the robot. In some embodiments, the cargo compartment may be modular and swappable to accommodate different cargo configurations or specialized equipment. Access to the cargo compartment may require multi-factor authentication, which may include RFID card detection and PIN code entry on the touchscreen user interface. In certain embodiments, additional authentication factors such as biometrics or SMS codes may be implemented for enhanced security.

In one or more embodiments, the robot system may include a camera system comprising a web camera for facilitating televisits and a depth camera for navigation and image classification tasks. The depth camera, such as the OAK-D model by Luxonis, may provide a 72-degree field of view from the front of the robot. This camera may serve a dual purpose-aiding in navigation and obstacle avoidance while also enabling AI-based image classification tasks when the robot is not actively navigating.

In one or more embodiments, the onboard computer system may be configured to run simultaneous localization and mapping (SLAM) algorithms for autonomous navigation. These algorithms may allow the robot to construct or update a map of an unknown environment while simultaneously keeping track of its location within it. The SLAM capability, combined with the sensor array including the 360-degree LIDAR, IMUs, and proximity sensors, may enable the robot to navigate dynamically changing environments and adapt its route in real-time to avoid obstacles.

In certain embodiments, the robot system may be configured to interact with building systems to facilitate autonomous navigation throughout a healthcare facility. For example, the robot may be equipped with sensors or communication receivers that enable it to send signals using Wi-Fi, infrared (IR), or radio frequency (RF) to interact with elevator systems. This capability may allow the robot to autonomously call and enter elevators to travel between floors.

In one or more embodiments, the modular cargo-carrying section of the robot may be customized to accommodate various components or hardware that enhance the robot's functionality. In some embodiments, this may include environmental sensors to monitor conditions such as temperature, humidity, air quality, or radiation levels as the robot moves through different areas of a facility. The data collected by these sensors may be logged and analyzed to identify potential issues or trends.

In one or more embodiments, alternative power sources may also be integrated into the modular cargo section in certain embodiments. For example, additional battery packs could be housed in the cargo area to extend the robot's operational time between charging sessions. In other embodiments, the cargo section could accommodate fuel cells or other emerging power technologies to provide extended runtime or rapid recharging capabilities.

In one or more embodiments, the robot system may include customizable cargo configurations to meet specific healthcare needs. For instance, temperature-controlled compartments could be integrated into the cargo area for transporting temperature-sensitive items like certain medications or laboratory samples. Other specialized compartments could be designed for secure transport of controlled substances or hazardous materials, incorporating additional locking mechanisms or containment features as required by regulations.

In some embodiments, the robot's AI-based image classification capabilities may be leveraged for inventory management tasks. The robot could be programmed to autonomously navigate storage areas, using its cameras and AI models to identify and count inventory items. This functionality could help healthcare facilities maintain accurate stock levels and automate reordering processes.

In one or more instances, the televisit capabilities of the robot may be further enhanced in certain embodiments. For example, the system could be integrated with healthcare platforms such as AI-based algorithms which capture and analyze image and other data or the electronic health record (EHR) systems, allowing healthcare providers to access patient information during remote consultations facilitated by the robot. Additionally, peripheral medical devices such as contactless patient monitoring devices could be connected to the robot, enabling more comprehensive remote examinations.

In one or more instances, to enhance the robot's ability to navigate in poorly lit areas, some embodiments may incorporate adaptive lighting systems. These could include adjustable LED arrays that automatically activate and adjust their intensity based on ambient light conditions detected by the robot's sensors. This feature may improve the robot's obstacle detection and navigation capabilities in dimly lit corridors or during nighttime operations.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application. While specific embodiments have been described, those skilled in the art will recognize modifications, adaptations, and variations that can be made without departing from the spirit of the invention. The scope of the invention is defined by the following claims.

What is claimed is:

1. An autonomous robot system for use in healthcare or laboratory facilities, comprising:
a mobile base with an omnidirectional drive system; a generally cylindrical body mounted on the mobile base; a secure cargo compartment integrated into the cylindrical body;
a plurality of sensors for autonomous navigation, environment monitoring and obstacle avoidance;
a touchscreen user interface mounted on an upper portion of the cylindrical body;
a camera system for facilitating televisits and image capture;
an electronic locking system to facilitate secure transport of cargo transport using a multifactor authentication;
an onboard computer system for controlling robot functions;
a wireless communication interface;
wherein the robot system is configured to autonomously navigate in space-restricted environments to securely transport cargo between locations, facilitate televisits, and perform AI-based image classification tasks.

2. The robot system of claim 1, wherein the omnidirectional drive system comprises three omni-wheels positioned 120° apart.

3. The robot system of claim 1, wherein the omnidirectional drive system comprises four mecanum wheels.

4. The robot system of claim 1, wherein the plurality of sensors comprises:
a 360-degree LIDAR sensor;
a depth camera;
an IMU sensor; and
a plurality of proximity sensors.

5. The robot system of claim 1, wherein access to the secure cargo compartment requires multi-factor authentication.

6. The robot system of claim 5, wherein the multi-factor authentication comprises RFID card detection and PIN code entry on the touchscreen user interface.

7. The robot system of claim 1, wherein the secure cargo compartment is modular and swappable to accommodate different cargo configurations.

8. The robot system of claim 1, wherein the camera system comprises:
a web camera for facilitating televisits; and
a depth camera for navigation and image classification tasks.

9. The robot system of claim 1, wherein the onboard computer system is configured to run AI models for image classification tasks including PPE compliance monitoring and inventory checking.

10. The robot system of claim 1, wherein the cylindrical body has a diameter of approximately 12.5 inches, enabling navigation in areas as narrow as 33 inches wide.

11. A method for autonomous operation of a healthcare robot, comprising:
receiving a task input via a user interface or wireless network connection;
authenticating a user for cargo access if the task involves cargo transport;
planning a navigation route to a specified destination;
autonomously navigating to the destination while avoiding obstacles exhibiting path deviation capabilities;
performing the specified task at the destination, wherein the task comprises at least one of:
delivering or retrieving cargo;
facilitating a televisit; or
capturing images for AI-based classification; and
returning to a home location or proceeding to a next task.

12. The method of claim 11, wherein autonomously navigating comprises:
detecting obstacles using a plurality of sensors;
using simultaneous localization and mapping (SLAM) algorithms in conjunction with its IMU, LIDAR and other sensors to determine the robot's position and update an environment map; and
adjusting the planned route to avoid detected obstacles.

13. The method of claim 11, wherein authenticating a user for cargo access comprises:
detecting an RFID card;
prompting for PIN entry on a touchscreen interface;
optional SMS authentication and unlocking the cargo compartment if both RFID and PIN authentication are successful.

14. The method of claim 11, wherein facilitating a televisit comprises:
navigating to a specified patient location;
initiating a video call using an onboard camera, display screen, speakers, microphone and conferencing software; and
enabling remote interaction between a healthcare provider and the patient.

15. The method of claim 11, wherein capturing images for AI-based classification comprises:
positioning the robot's camera to capture a specified field of view; acquiring image data;
processing the image data using pre-trained AI models stored on the robot's onboard computer; and
outputting classification results.

16. A healthcare robot system, comprising:
a mobile base less than 14 inches in diameter;
an omnidirectional drive system integrated into the mobile base;
a cylindrical body mounted vertically on the mobile base;
a secure cargo compartment integrated into the cylindrical body;
a touchscreen user interface mounted on an upper portion of the cylindrical body;
a 360-degree LIDAR sensor for obstacle detection;
a depth camera for navigation and image capture;
a plurality of proximity sensors;
a web camera for televisits;
an onboard computer system; and
a wireless network interface;
wherein the robot system is configured to:
autonomously navigate in spaces as narrow as 33 inches wide;
securely transport cargo between locations using multi-factor authenticated access;
facilitate televisits between remote healthcare providers and patients; and
perform AI-based image classification tasks when not actively navigating.

17. The robot system of claim 16, wherein the omnidirectional drive system comprises three omni-wheels positioned 120° apart.

18. The robot system of claim 16, wherein the secure cargo compartment is modular and swappable.

19. The robot system of claim 16, wherein the onboard computer system is configured to run simultaneous localization and mapping (SLAM) algorithms for autonomous navigation.

20. The robot system of claim 16, further comprising a fleet management system for coordinating multiple robot units within a healthcare facility.
